SEQUENCE LISTING (15 sequences)

SEQ ID NO: 1 - protein, 7 aa, Artificial Sequence
  Synthetic Construct
HPHHHHE

SEQ ID NO: 2 - DNA, 57 bp, Artificial Sequence
  Synthetic Construct
gcatccgccccaaagcttgaagaaggtgaattttcagaagcacgcgtagatatcgtg

SEQ ID NO: 3 - protein, 19 aa, Artificial Sequence
  Synthetic Construct
ASAPKLEEGEFSEARVDIV

SEQ ID NO: 4 - DNA, 16 bp, Artificial Sequence
  Synthetic Construct
atccgccccaaagctt

SEQ ID NO: 5 - DNA, 16 bp, Artificial Sequence
  Synthetic Construct
acgatatctacgcgtg

SEQ ID NO: 6 - DNA, 36 bp, Artificial Sequence
  Synthetic Construct
atccgccccaaagcttnnbgcacgcgtagatatcgt

SEQ ID NO: 7 - DNA, 39 bp, Artificial Sequence
  Synthetic Construct
atccgccccaaagcttnnbnnbgcacgcgtagatatcgt

SEQ ID NO: 8 - DNA, 42 bp, Artificial Sequence
  Synthetic Construct
atccgccccaaagcttnnbnnbnnbgcacgcgtagatatcgt

SEQ ID NO: 9 - DNA, 45 bp, Artificial Sequence
  Synthetic Construct
atccgccccaaagcttnnbnnbnnbnnbgcacgcgtagatatcgt

SEQ ID NO: 10 - DNA, 48 bp, Artificial Sequence
  Synthetic Construct
atccgccccaaagcttnnbnnbnnbnnbnnbgcacgcgtagatatcgt

SEQ ID NO: 11 - DNA, 51 bp, Artificial Sequence
  Synthetic Construct
atccgccccaaagcttnnbnnbnnbnnbnnbnnbgcacgcgtagatatcgt

SEQ ID NO: 12 - DNA, 54 bp, Artificial Sequence
  Synthetic Construct
atccgccccaaagcttnnbnnbnnbnnbnnbnnbnnbgcacgcgtagatatcgt

SEQ ID NO: 13 - DNA, 57 bp, Artificial Sequence
  Synthetic Construct
atccgccccaaagcttnnbnnbnnbnnbnnbnnbnnbnnbgcacgcgtagatatcgt

SEQ ID NO: 14 - DNA, 60 bp, Artificial Sequence
  Synthetic Construct
atccgccccaaagcttnnbnnbnnbnnbnnbnnbnnbnnbnnbgcacgcgtagatatcgt

SEQ ID NO: 15 - DNA, 63 bp, Artificial Sequence
  Synthetic Construct
atccgccccaaagcttnnbnnbnnbnnbnnbnnbnnbnnbnnbnnbgcacgcgtagatatcgt

(19) United States
(12) Patent Application Publication
(10) Pub. No.: US 2005/0054000 A1
(43) Pub. Date: Mar. 10, 2005

(54) Fv constructs with an influenceable affinity for a substance that is to be linked

(76) Inventor: Stefan Dubel, Dossenheim (DE)

Correspondence Address: INTELLECTUAL PROPERTY / TECHNOLOGY LAW, PO BOX 14329, RESEARCH TRIANGLE PARK, NC, 27709, US

(21) Appl. No.: 10/344,447

(22) PCT Filed: Aug. 10, 2001
(86) PCT No.: PCT/DE01/03098

(30) Foreign Application Priority Data
Aug. 11, 2000 (DE) 10040046.9
Sep. 5, 2000 (DE) 10044002.9

(51) Int. Cl. G01N 33/53; C07K 16/28

(52) U.S. Cl. 435/7.1; 530/388.22; 530/391.1

(57) ABSTRACT

The present invention relates to Fᵥ constructs having an affinity that can be influenced for a substance to be linked, wherein the Fᵥ constructs have peptides linked to the variable regions and containing binding sites for effector molecules. The invention also relates to a method of producing a single-chain Fᵥ construct and the use of an Fᵥ construct according to the invention for enriching and purifying substances.

The present invention relates to Fᵥ constructs whose affinity for a substance to be linked can be influenced, e.g. the linkage between the Fᵥ construct and the substance can be broken again without the two being impaired considerably. The invention also relates to a method of producing such an Fᵥ construct and its use for enriching and purifying substances.

Fᵥ constructs are usually constructs from the variable regions of an antibody (referred to below as Fᵥ antibody constructs) or a T-cell receptor (referred to below as Fᵥ T-cell receptor constructs), the variable regions serving as binding sites for a substance to be linked, e.g. an antigen or a ligand. In this connection, a binding site of an Fᵥ antibody construct may be formed of a V_{H} region and a V_{L} region, of two V_{H} regions when the binding site is derived from a camel antibody, for example, or of two V_{L} regions when the binding site is derived from a Bence Jones protein. A binding site of an Fᵥ T-cell receptor construct may be formed of an α region and a 1 region. Fᵥ constructs are often available as single chains, i.e. the variable regions are linked with one another via peptide linkers. Such Fᵥ constructs are referred to as scFᵥ constructs. They may be monospecific, bispecific or oligospecific, i.e. they have binding sites for one, two or more different substances.

Fᵥ antibody constructs are often used for enriching and purifying substances, e.g. proteins, receptors, viruses, etc. This is effected by affinity chromatography methods, for example, by which the Fᵥ antibody constructs are bound to a matrix, e.g. a chromatographic column, on which the solutions that contain said substances are placed so that the substances may bind to the corresponding Fᵥ antibody constructs. The enriched substances are then obtained by elution. This step is usually carried out under stringent conditions, e.g. highly molar salt solutions, solutions having a strongly acidic or basic pH or by means of chaotropic/denaturing agents, in particular when the affinity of the Fᵥ antibody constructs for the substances is high. As a result, both the Fᵥ antibody constructs and the substances are impaired considerably, which often leads to the fact that the Fᵥ antibody constructs and thus the chromatographic column can no longer be used and the substances lose their activity.

It is thus the object of the present invention to provide a product by which substances bound to Fᵥ antibody constructs can be removed again without the above drawbacks occurring for the Fᵥ antibody constructs and/or the substances.

According to the invention this is achieved by the subject matters defined in the claims.

The present invention is based on applicant's findings that the affinity of an Fᵥ construct for a substance to be linked can be influenced, so that e.g. the linked substance can be removed again by moving the variable regions forming the binding site at the Fᵥ constructs against one another. He also realized that no above elution conditions are necessary for this and that the Fᵥ constructs and the substances are not impaired considerably, e.g. as regards their reusability and activity. He rather found that the opposite movement of the variable regions, e.g. by binding effector molecules, such as ions or antibodies, to corresponding binding sites can be effected on peptides linked to the variable regions. The binding properties of the Fᵥ constructs of the present invention are changed subsequently, i.e. after the concluded production and folding, but not during the manufacturing process. This means that this is an allosteric modulation to already fully produced and folded antibody fragments. Reference is made to the below examples.

According to the invention, these applicant's findings are utilized to provide an Fᵥ construct having an affinity which can be influenced for a substance to be linked, the Fᵥ construct having peptides linked to the variable regions and containing binding sites for effector molecules.

The employed term “Fᵥ construct” relates to an Fᵥ construct whose variable regions include peptides having binding sites for effector molecules. It may be favorable for the variable regions to be linked to one another via the peptides, it being possible to effect this e.g. via a disulfide bridge or a peptide bond. The peptides are preferably linked to one another via a peptide bond, so that the Fᵥ construct is present as a single-chain (sc)Fᵥ construct. The Fᵥ construct may be monospecific, bispecific or oligospecific, i.e. it may have binding sites for one or several substances, such as proteins, receptors, antibodies, ligands, viruses, synthetic compounds, etc. An Fᵥ construct is preferably an Fᵥ antibody construct or an Fᵥ T-cell receptor construct. In this connection, it may be favorable for these Fᵥ constructs to also have constant regions. It is particularly preferred for an Fᵥ construct to have binding sites for an adeno-associated virus (AAV) and/or a capsid thereof. The Fᵥ construct may also be a polymer, e.g. a dimer or tetramer, of an scFᵥ construct, the polymer being formed by juxtaposition of several scFᵥ constructs.

The employed term “affinity which can be influenced of the Fᵥ construct for a substance to be linked” relates to both the increase and the reduction of such an affinity, without the Fᵥ construct and the substance being impaired considerably, e.g. as regards their reusability and/or activity. In particular, influencing the affinity of the Fᵥ construct for the substance to be linked is understood to mean the removal of the linked substance from the Fᵥ construct, which is of major importance e.g. for the enrichment and purification of a substance by means of affinity chromatography.

The employed term “peptides having binding sites for effector molecules” relates to (poly)peptides of any type and origin, which may be linked to variable regions and may have binding sites for effector molecules. The peptides may be peptides which occur in nature and into which such bindings sites are introduced, where appropriate, or they may be synthetic peptides. It may be favorable for the peptides of the individual variable regions to be linked to one another, this being effected via a disulfide bridge or a peptide bond, for example. The effector molecules to be mentioned are any effector molecules effecting an opposite movement of the variable regions of an Fᵥ construct by linking to their binding sites available within the peptides, one or more of said binding sites being available per peptide. The effector molecules are preferably antibodies or ions, e.g. bivalent or trivalent ions, in particular metal ions, with nickel, copper and zinc ions being particularly preferred. The binding sites for antibodies to be mentioned are their epitopes on corresponding antigens, whereas in particular zinc fingers and His repeats have to be mentioned as binding sites for metal ions. The binding site suited as an effector molecule for nickel ions preferably has the amino acid sequence HPHHHHE. It is particularly preferred for the effector molecules to be easily removable from the binding sites on the peptide linker again, which may be effected e.g. in the case of metal ions such as nickel, copper, and zinc ions, via chelating agents, such as EDTA or EGTA. Reference is also made to the below examples.

In a preferred embodiment an above Fᵥ construct is available in connection with a matrix. It may be any solid carrier to which the Fᵥ construct can be linked by means of common methods, e.g. via unpaired cysteine residues or the avidin/streptavidin method. The carrier is preferably a chromatographic column so that the Fᵥ construct is particularly suited for affinity chromatography.

An Fᵥ construct according to the invention can be produced by various methods. For example, if an scFᵥ construct according to the invention shall be produced, it may be favorable to use a method in which a DNA coding for variable regions, the regions being linked to one another via binding sites for peptides having effector molecules, is inserted in an expression vector and expressed via the latter in cells. The Fᵥ construct according to the invention can then be isolated from the cells or the cell supernatant and purified. It is favorable for the Fᵥ construct to be presented via a phage display expression vector, e.g. pSEX81 on the surface of phages so as to facilitate its identification. Reference is made to the below examples. As to the expressions “Fᵥ construct” and “peptides having binding sites for effector molecules” reference is made to the above explanations. Reference is also made to Maniatis, T. et al., Molecular Cloning, A Laboratory Manual, Cold Spring Harbor Laboratory 1982, by way of supplement.

A further subject matter of the present invention relates to a kit comprising:
(a) an Fᵥ construct according to the invention,
(b) a DNA according to the invention, in particular an expression vector according to the invention, and
(c) common auxiliary agents, such as buffers, solvents and controls.

One or several representatives of the individual components may be available.

The present invention provides an Fᵥ construct whose affinity for a substance to be linked can be influenced, so that e.g. the removal of the linked substance can be achieved again. For this it is typical that the influence of the affinity is mild enough for the Fᵥ construct and the substance not to be impaired considerably so that they can be reused and remain active. Thus, the present invention provides a possibility which is very well suited to enrich and purify substances, in particular sensitive substances having a biological function, such as proteins, receptors, ligands and viruses, such as viruses suited for gene therapy, in particular AAV.

The invention is explained by the below examples.

### EXAMPLE 1

### Production of an Fᵥ Antibody Construct According to the Invention

The scFᵥ antibody construct scFᵥ215 binds to Drosophila RNA polymerase (Kontermann et al., (1995), Biol. Chem. 376, 473-481). The cloning into the expression vector pOPE101 (Genebank No.: Y14585) was effected by means of the method according to Breitling & Dübel, Methods in Molecular Medicine, Vol. 13, (1997), ed. U. Reischl, Humana Press Inc., Totowa, N.Y. 581-591. Following the addition of 50 μM IPTG to the culture medium of the bacteria, this expression vector enables a production of the scFᵥ antibody construct in the periplasm of the bacteria from which it can be isolated. By this cloning, an NcoI restriction site and a HindIII restriction site were introduced into the V_{H} region and an MluI restriction site and a NotI restriction site were introduced at the ends of the respective V regions. The DNA coding for the scFᵥ215 antibody construct was then cloned into the E. coli phage display vector pSEX81 (Genebank No.: Y14584) via the restriction sites NcoI and NotI so as to obtain pSEX81-215(Yol). This expression vector enables the phage display of antibody constructs by expressing the scFᵥ antibody construct as an aminoterminal fusion protein with the minor coat protein (pIII) of a filamentous M13 group phage thus achieving a surface presentation of the scFᵥ antibody construct. The sequence identity with the planned sequence was checked by DNA sequencing of the insert.

Furthermore, the linker between the V_{H} and V_{L} regions of the scFᵥ215 antibody construct was replaced by peptides coding for a plurality of different sequences. These peptides were constructed at 10 different lengths by preparing peptide libraries from oligonucleotides which in place of the DNA coding for the linker between the V_{H} and V_{L} regions from pOPE101-215(Yol) were inserted in this vector. For this purpose, the following steps were taken:

10 different mixtures (Wobbel libraries) of oligonucleotides were synthesized (LINKLIB6-LINKLIB15) which coded for 10 different peptide lengths (below Table 1). In these oligonucleotides, 6-15 codons (NNB, wherein N represents all bases and B represents the bases G, C, T) were randomized. The randomized regions were also provided respectively at the 5′ end of the oligonucleotide with a HindIII restriction site and the 10 V_{H} region nucleotides ahead thereof, which follow from the sequence of the scFᵥ antibody construct, and at the 3′ end with an MluI restriction site and the then following 10 V_{L} region nucleotides which follow from the sequence of the scFᵥ antibody construct. Two further oligonucleotide primers (RANFOR and RANBACK) were prepared for the PCR amplification of these 10 oligonucleotide libraries, the sequence of the first oligonucleotide primer corresponding to the sequence of the 5′ end of the randomized oligonucleotide in so far as the sequence does not yet contain any Wobbel codons and the sequence of the second oligonucleotide primer corresponds to the counterstrand of the 3′ end of the randomized oligonucleotide, starting from the point where it no longer contains any Wobbel codons.

TABLE 1

Oligonucleotides for preparing the peptide linker
libraries

```
Orig. Vector pSEX81-215(Yol)
      VH HindIII Linker MluI EcoRV VL
...GCATCCGCCCCAAAGCTTGAAGAAGGTGAATTTTCAGAAGCACGCGTAGATATCGTG
        A S A P K L E E G E F S E A R V D I V
Oligonukleotid RANFOR
5′-ATCCGCCCCAAAGCTT
Oligonukleotid RANBACK
5′-ACGATATCTACGCGTG
Oligonukleotid LINKLIB6
5′-ATCCGCCCCAAAGCTTNNBGCACGCGTAGATATCGT
Oligonukleotid LINKLIB7
5′-ATCCGCCCCAAAGCTTNNBNNBGCACGCGTAGATATCGT
Oligonukleotid LINKLIB8
5′-ATCCGCCCCAAAGCTTNNBNNBNNBGCACGCGTAGATATCGT
Oligonukleotid LINKLIB9
5′-ATCCGCCCCAAAGCTTNNBNNBNNBNNBGCACGCGTAGATATCGT
Oligonukleotid LINKLIB10
5′-ATCCGCCCCAAAGCTTNNBNNBNNBNNBNNBGCACGCGTAGATATCGT
Oligonukleotid LINKLIB11
5′-ATCCGCCCCAAAGCTTNNBNNBNNBNNBNNBNNBGCACGCGTAGATATCGT
Oligonukleotid LINKLIB12
5′-ATCCGCCCCAAAGCTTNNBNNBNNBNNBNNBNNBNNBGCACGCGTAGATATCGT
Oligonukleotid LINXLIB13
5′-ATCCGCCCCAAAGCTTNNGNNBNNBNNBNNBNNBNNBNNBGCACGCGTAGATATCGT
Oligonukleotid LINKLIB14
5′-ATCCGCCCCAAAGCTTNNBNNBNNBNNBNNBNNBNNBNNBNNBGCACGCGTAGATATCGT
Oligonukleotid LINKLIB15
5′-ATCCGCCCCAAAGCTTNNBNNBNNBNNBNNBNNBNNBNNBNNBNNBGCACGCGTAGATATCGT
```

By means of RANFOR and RANBACK as primers and the randomized 10 different mixtures (Wobbel libraries) of oligonucleotides (LINKLIB6-LINKLIB15) as a template 10 corresponding PCR reactions were carried out with 15 cycles each, which resulted in an amplification of the DNA sequences coding for the peptides. The PCR products were purified by means of agarose gel electrophoresis. Following phenolization, restriction digestion using MluI and HindIII and another purification by means of gel electrophoresis, the PCR products were cloned into the pSEX81-215(Vol) expression vector which was also excised using MluI and HindIII. For this purpose, the ligation products were electro-transformed by means of electroporation into 50 μl competent E. coli X11blue cells each and plated onto culture medium agar plates with ampicillin for the purpose of selection and grown at 37° C. overnight. The resulting clones were then taken up in 500 μl LB broth and amplified, and the formation of phages which produce Fᵥ antibody constructs induced by adding helper phages (Welschof et al., Proc. Natl. Acad. Sci. U.S.A. 94, (1997), 1902-1907). Each phage carried respectively the V_{H} and V_{L} regions of the scFᵥ215 antibody construct, which were linked to the different peptides (10). In each case, 10¹³ phages of these 10 different phage suspensions were placed in a polystyrene tube which had first been coated with the antigen 215-βGal, i.e. a fusion protein purified from E. coli inclusion bodies and made from β-galactosidase and the eptiope peptide of the scFᵥ215 antibody construct (Kontermann et al., (1995), supra) and thereafter with 2% milk powder (as a block solution) (“panning”). Following incubation at room temperature for 6 hours, the phage suspensions were sucked off and the polystyrene tube was washed 20 times using PBS to remove unbound phages. Thereafter, elution of the bound phages was carried out with increasing concentrations of nickel chloride in PBS (0.1, 0.5; 0.7; 1, 1.5; 2, 3; 4.5 M). In order to neutralize the nickel, the eluates were mixed with Chelating Sepharose suspended in PBS (Pharmacia company) and mixed with plating bacteria (E. coli X11blue) 15 minutes later. Following infection at 37° C. for one hour, the plating bacteria were plated onto ampicillin selection medium. The resulting clones were respectively induced again to form phages producing Fᵥ antibody constructs. Three further runs of “panning” were carried out as described above. However, in that case a concentration was used for the elution at which the eluate of the primary elution was obtained. After a total of 4 panning runs, colonies were found in 26 of 90 assays, over 0.5 M NiCl being present at these elution concentrations. Of the 26 plates, 12 were selected from which 10 individual clones each were picked and induced to form phages which produce Fᵥ antibody constructs. The resulting 120 individual clones were investigated in a phage ELISA in 96-well ELISA plates, 215-βGal being used as the antigen and the detection being made with the monoclonal antibody B62-FE2 (Michael, B. et al., (1994), J. 1 mm. Methods 171, 103-109). The original vector pSEX81-215(Yol) served as a control and two wells were used for each clone, one without and the other with 1 M NiCl during the incubation of the phages with the antigen. Of the 120 individual clones, 63 showed a positive reaction (=antigen binding), and of the latter, 45 could be competed with nickel. The 45 competable clones were pooled and again packed as phages which were then again subjected to a panning run in which the elution was made with 0.5 M NiCl in PBS. Following reinfection and amplification in plating bacteria 12 colonies of the eluted phages were picked and their peptide linker between the V_{H} and V_{L} regions was sequenced on the DNA level. All of the 12 phages had a uniform DNA sequence coding for the peptide linker HPHHHHE.

### EXAMPLE 2

### Use of the Fᵥ Antibody Construct According to the Invention for Enriching and Purifying a Substance

Following the recloning of an scFᵥ215 antibody construct including the peptide HPHHHHE according to Example 1 in pOPE51, the scFᵥ antibody construct was produced in E. coli, purified and covalently bonded to an affinity chromatography column via the unpaired cysteine near the carboxy terminus. A purified, active fusion protein from the M13 phage protein was placed on this column pIII, whose last 24 amino acid residues were deleted and replaced by the epitope peptide of the scFᵥ215 antibody construct. The subsequent elution was carried out with a gradient of 0-1 M NiCl in PBS. The fusion protein was eluted from a concentration of 0.22 M, the peak being at 0.4 M. The yield was 72%. Dialysis in PBS was carried out to remove the nickel chloride. The specific activity of pIII (measured in the infection inhibition test on F-Pili of E. coli) corresponded to that of a pIII used as a control and not placed on the affinity chromatography column. These results were confirmed several times by means of experiments in which the same affinity chromatography column was used.

Hence this shows that the affinity of an Fᵥ construct according to the invention for a substance to be linked can be influenced, i.e. the substance can e.g. be removed again without considerable impairment of the Fᵥ construct, e.g. as regards its reusability, and the substance, e.g. as regards its activity.

1. An Fᵥ construct having an affinity which can be influenced for a substance to be linked, the Fᵥ construct having peptides linked to the variable regions and containing binding sites for effector molecules, wherein the variable regions are linked to one another via peptides.

2. The Fᵥ construct according to claim 1, wherein the Fᵥ construct is a single-chain Fᵥ construct.

3. The Fᵥ construct according to claim 1 or 2, wherein the Fᵥ construct is an Fᵥ antibody construct.

4. The Fᵥ construct according to claim 1 or 2, wherein the Fᵥ construct is an Fᵥ T-cell receptor construct.

5. The Fᵥ construct according to any of claims 1 to 4, wherein the Fᵥ construct is monospecific.

6. The Fᵥ construct according to any of claims 1 to 4, wherein the Fᵥ construct is bispecific.

7. The Fᵥ construct according to any of claims 1 to 6, wherein the effector molecules are ions.

8. The Fᵥ construct according to claim 7, wherein the ions are bivalent or trivalent ions.

9. The Fᵥ construct according to claim 7 or 8, wherein the ions are metal ions.

10. The Fᵥ construct according to claim 9, wherein the metal ions are nickel, copper or zinc ions.

11. The Fᵥ construct according to claim 9 or 10, wherein the binding sites for the metal ions are zinc fingers or HIS repeats.

12. The Fᵥ construct according to any of claims 1 to 6, wherein the effector molecules are antibodies.

13. The Fᵥ constructs according to any of claims 1 to 12, wherein the Fᵥ construct is available in connection with a matrix.

14. The Fᵥ construct according to claim 13, wherein the matrix is a chromatographic column.

15. The Fᵥ construct according to any of claims 1 to 14, wherein the substance to be linked is a protein, a receptor, a ligand, a virus or a synthetic compound.

16. A method of producing the Fᵥ construct according to any of claims 2 to 12, wherein a DNA coding for variable regions, the regions being linked to one another via binding sites for peptides having effector molecules, is inserted in an expression vector and expressed via the latter in cells.

17. Use of the Fᵥ construct according to any of claims 1 to 15 for enriching and purifying substances.

18. Use according to claim 15, wherein the enrichment and purification are made on the basis of an affinity chromatography.
